(19) United States
(12) Patent Application Publication
(10) Pub. No.: US 2006/0263365 A1
(43) Pub. Date: Nov. 23, 2006

(54) Method of immunomodulation

(76) Inventor: Derek Hart, Queensland (AU)

Correspondence Address: SCULLY SCOTT MURPHY & PRESSER, PC, 400 GARDEN CITY PLAZA, SUITE 300, GARDEN CITY, NY, 11530, US

(21) Appl. No.: 10/523,756

(22) PCT Filed: Aug. 8, 2003
(86) PCT No.: PCT/AU03/01011

§ 371 Date: Jun. 29, 2006

(30) Foreign Application Priority Data
Aug. 8, 2002 (AU) 2002950658

(51) Int. Cl. A61K 39/395

(52) U.S. Cl. 424/146.1

(57) ABSTRACT

The present invention relates generally to a method for modulating the immuno-activity of an antigen-presenting cell and agents useful therefor. More particularly, the present invention relates to a method for preventing or down-regulating one or more functional activities of a dendritic cell. The present invention further provides antibodies and, in particular, monoclonal antibodies, which interact specifically with epitopes present on the surface of dendritic cells, resulting in depletion, down-regulation or destruction of targeted dendritic cell in vivo or in vitro. The instant invention further provides a method for modulating an immune response in a subject and, in particular, for down-regulating the immuno-activity of an allogeneic immuno-competent graft and/or the immune response of a recipient of a solid organ transplant. The ability to modulate dendritic cell immuno-activity may be useful, inter alia, in a range of immuno-therapeutic and immuno-prophylactic treatments that benefit from immune suppression.

### FIELD OF THE INVENTION

The present invention relates generally to a method for modulating the immuno-activity of an antigen-presenting cell and agents useful therefor. More particularly, the present invention relates to a method for preventing or down-regulating one or more functional activities of a dendritic cell. The present invention further provides antibodies and, in particular, monoclonal antibodies, which interact specifically with epitopes present on the surface of dendritic cells, resulting in depletion, down-regulation or destruction of targeted dendritic cell in vivo or in vitro. The instant invention further provides a method for modulating an immune response in a subject and, in particular, for down-regulating the immuno-activity of an allogeneic immuno-competent graft and/or the immune response of a recipient of a solid organ transplant. The ability to modulate dendritic cell immuno-activity may be useful, inter alia, in a range of immuno-therapeutic and immuno-prophylactic treatments that benefit from immune suppression.

### BACKGROUND OF THE INVENTION

Reference to any prior art in this specification is not, and should not be taken as, an acknowledgment or any form of suggestion that this prior art forms part of the common general knowledge in any country.

Dendritic cells (DC) are potent cellular activators of primary immune responses (Hart, Blood 90: 3245-3287, 1997). Immature myeloid DC in non-lymphoid organs react to, endocytose and process antigens and migrate via blood and lymph to T cell areas of lymphoid organs. Here, the mature cells present foreign peptide complexed to MHC Class II to T cells and deliver unique signals for T-cell activation (immuno-stimulation). They also stimulate B lymphocytes and NK cells. DC undergo differentiation/activation during this process, lose their antigen-capturing capacity and become mature, immuno-stimulatory DC that trigger naïve T-cells recirculating through the lymphoid organs. The lymphoid DC subset may have a different migration pathway and although capable of stimulating allogeneic and autologous T-lymphocytes they have been suggested to have a regulatory function (Grouard et al., J. Exp. Med. 185: 1101-1111, 1997). As part of the differentiation/activation process, DCs up-regulate certain relatively selectively-expressed cell surface molecules such as the CMRF-44 and CD83 antigens. DC in the thymus and DCs that do not have an activated co-stimulating phenotype probably contribute to central and peripheral tolerance.

Allogeneic transplantation involves the transfer of material from a host to a recipient. In this process, many foreign antigens are introduced into a host and an immune response results when these foreign antigens are detected by the host's immune system. Initially, an immune response involves interactions between the antigen and antigen-presenting cells (APC) such as dendritic cells. Interstitial donor DC in heart and kidney contribute to (direct) recipient T lymphocyte sensitization to all antigens but recipient DC, after migrating into the donor tissue, can also stimulate (indirect) alloantigen sensitization of recipient T-lymphocytes. Depletion of heart and kidney and pancreatic islet DC appears to prolong allograft survival. Interestingly, during liver transplantation, donor leucocytes, which may include non-activated dendritic cells, appear to generate allogeneic tolerance. DC are also predicted to contribute to both acute and chronic Graft Versus Host Disease (GVHD), the major life threatening complication of allogeneic bone marrow transplantation (BMT). Blood DC counts change during acute GVHD and recent data have suggested that the DC subset constitution of the allogeneic stem cell preparation might relate to GVHD outcome. Recent evidence from a mouse model suggests that host APC contribute to the acute GVHD. DC may in certain circumstance prevent acute GVHD.

Monoclonal antibodies (mAb) which act at the level of the responder T lymphocyte have been investigated as therapeutic immunosuppression agents in allogeneic transplantation. The CD3 reagent OKT3 (Orthoclone, Cilag) is used routinely to treat acute renal allograft rejection. Campath 1 (CD52) and its variants have been used in solid organ transplant and BMT. More recent attempts to suppress acute GVHD have involved the antibody ABX-CBL (CD147) (Deeg et al., Blood 98: 2052-2058, 2001) and anti-IL-2R mAb Daclizumab (Cahn et al., Transplantation 60: 939-942, 1995). Attempts to interfere with the interaction of the responder T-lymphocyte and an APC have focused on antibodies directed against the co-stimulator molecules CD40, CD80 and CD86 or their ligands. Animal studies suggest that blockade of co-stimulator molecules on DC and other APC induces T cell anergy and prolongation of solid organ grafts (Koenen and Joosten, Blood 95: 3153-3161; 2000, Kirk et al., Nat. Med. 5: 686-693, 1999; Kirk et al. Proc Natl Acad Sci USA 94: 8789-8794, 1997). The use of CD80, CD86 and CD28 blocking agents prevents acute GVHD in mice (Blazar et al., Blood 85: 2607-2618, 1995) and in vitro blockage of allogeneic responses in allogeneic stem cell preparations has been used in clinical BMT with initial encouraging results (Gribben et al., Blood 87: 4887-4893, 1996). The use of a reagent that was more selective at targeting differentiated/activated DC might be advantageous.

In humans, at least two populations of DC, the immature myeloid DC and the plasmacytoid DCs, have been identified based on differential expression of CD11c (O'Doherty et al., J Exp Med 178: 1067, 1993; O'Doherty et al., Immunol 82: 487, 1994) More recent studies have shown that CD11c⁻ DC have a different phenotype and express higher amounts of CD123, and have a morphology and function distinct from CD11c⁺ DC (Grouard et al., J Exp Med. 185: 1101-1111, 1997). These two subsets are denoted as myeloid lineage CD11c⁺ DC and plasmacytoid CD123⁺ DC. It is thought unlikely that there is a direct developmental relationship between them (Robinson et al., Eur J Immunol 29: 2769, 1999).

Theoretically, mAb directed at DC administered to the recipient of a solid organ graft would deplete donor DC (i.e. direct) as well as recipient DC (indirect) as they enter the circulation and initiate antigen presentation pathways. Other donor leucocytes may have immunomodulatory capacity. DC depletion therapy might then be ceased after a short period, allowing tolerance to emerge. Depleting recipient DC may be more efficacious than disrupting co-stimulator pathways. Investigation of this concept has been delayed, however, by the absence of suitable DC reagents. CMRF-44 mAb is an antibody specific for DC and is used for the identification and isolation of human blood DC (Fearnley et al., Blood 89: 3708-3716, 1997). The latter authors have shown that the epitope for CMRF-44 mAb (i.e. CMRF-44 Ag) is expressed early in the differentiation of DC from circulating precursor cells.

Given the importance of dendritic cells in the overall immuno-potential of an individual, there is a need to identify agents, which can facilitate modulation of DC activity.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention is predicated in part on the determination that a cell-surface activation molecule may act as a target for agents, the binding of which, results in disablement and/or eventual destruction of the cell. In particular, it has been shown that CMRF-44 mAb is capable of initiating lysis of antigen presenting cells such as DC. More particularly, CMRF-44 is capable of acting as an immuno-suppressive agent, by down-regulating DC function. Thus, the present invention provides reagents useful for the down-regulation of activated DC, and a method for the suppression of an immune response useful inter alia for the reduction or prevention of allogeneic graft rejections, graft versus host disease, and the amelioration of certain auto-immune inflammatory interactions, such as rheumatoid arthritis.

The present invention, therefore, contemplates a method for modulating the immuno-activity of an antigen-presenting cell (APC) by contacting the APC with an effective amount of an agent which couples, binds or otherwise associates with a cell-surface activation molecule and in turn prevents, inhibits or otherwise down-regulates one or more functional activities of the APC.

Generally, the APC is a DC.

Preferably, the DC is a myeloid DC and, in a particularly preferred embodiment, belongs to the CD11c⁺ DC sub-population.

In a preferred embodiment, the agent comprises a monoclonal antibody such as, for example, CMRF-44, or a derivative, fragment, homolog, analog or chemical equivalent or mimetic thereof and the cell-surface activation molecule is a molecule or a derivative, fragment, homolog, analog or chemical equivalent or mimetic thereof, expressed on the surface of a DC and which interacts with CMRF-44 antibody.

The present invention is further directed to a method for modulating an immune response in a subject by administering to the subject an effective amount of an agent which couples, binds or otherwise associates with an antigen presenting cell's surface activation molecule (e.g. a DC surface molecule which interacts with CMRF-44) which in turn prevents, inhibits or otherwise down-regulates one or more functional activities of the APC.

The agent of the present invention may also be used to down-regulate the immuno-activity of an immuno-competent graft such as a bone marrow graft.

Another aspect of the present invention contemplates a method for the prophylactic and/or therapeutic treatment of a condition characterized by the aberrant, unwanted or otherwise inappropriate immuno-activity of an immuno-competent graft by contacting the graft with an effective amount of the agent or a derivative, homolog, analog, chemical equivalent or mimetic thereof which prevents, inhibits or otherwise down-regulates the inappropriate immuno-activity of the graft.

The present invention further extends to pharmaceutical compositions and formulations comprising the agent for use in conjunction with the instant methods, and to the use of such agents in the manufacture of a pharmaceutical composition or formulation.

### BRIEF DESCRIPTION OF THE FIGURES

FIG. 1 are graphical representations showing examples of CMRF-44 expression in cultured human leukocytes. (A) PMBC (activated DC are defined as PE⁻ FITC⁺ cells, in lower right quadrant), (B) purified Lin⁻ PBMC cultured overnight with GM-CSF and IL-4, (C) CD11c⁺ Lin⁻ PBMC cultured as in B, and (D) CD123^{hi} Lin⁻ PBMC cultured as in B. In A, the quadrant positions were determined by negative control staining. In B-D, the left hand line represents IgM negative control staining.

FIG. 2 are graphical representations showing CMRF-44-specific complement-mediated DC lysis occurs in cultured human PBMC. The combination of CMRF-44 and autologous human scrum (AS) deplete CD83⁺ DC. Treatments=(A) AS only, (B) CMRF-44 mAb only, (C) CMRF-44+AS, (D) negative control IgM+AS. Lower right quadrants show percentage of DC in treated cultured PBMC.

FIG. 3 are graphical representations showing Lin⁻ DC survival is improved with GM-GSF and IL-3 present during overnight culture. (A) Cell death analyzed by PI/Annexin-V labeling after overnight culture with or without the addition of GM-CSF/IL-3. (B) Example of Lin⁻ DC, in live forward/side scatter gate showing improved yield of CMRF-44⁺ cells after culture with GM-CSF+IL-3 (left-hand curves+IgM negative control).

FIG. 4 are graphical representations showing CMRF-44-specific complement-mediated lysis of DC within a cultured purified human DC (Lin⁻ cell) preparation. The effect on the percentage of CD11c⁺ HLA-DR⁺ cells (dot plots, upper right quadrants) and on the percentage of dead 7-AAD⁺ cells (histograms) after treatment with (A) medium alone, (B) 1:2 v/v AS alone, and (C) 20 ug/ml CMRF-44 and AS combined is shown.

FIG. 5 are graphical representations showing examples of CMRF-44 specific complement mediated lysis of cultured CD11c⁺ and CD123 ^{hi} DC sort purified from a Lin⁻ preparation. (A, B) HLA-DR⁺ CD11c⁺ DC treated with autologous human serum (AS) and either (A) negative control IgM, or (B) CMRF-44 mAb. (C, D) HLA-DR⁺ CD123^{hi} DC treated as in A, B. The same initial numbers of cells were treated in each case and the same number of TruCount beads were acquired for each dot plot.

FIG. 6 are graphical representations showing the primary proliferative KLH response induced by PBMC is reduced by treatment with CMRF-44 and complement. Treatments=CMRF-44 mAb and AS, (shaded bars) or CMRF-44 and HIAS (Black bars). (*−p<0.05 Student's t-test, error bars±2SE). Two independent experiments (A and D) are shown.

FIG. 7 are graphical representations showing recall proliferative response to tetanus toxoid (TT) induced by PBMC is reduced by treatment with CMRF-44 and complement. Treatments CMRF-44 mAb and AS (shaded bars) or CMRF-44 and HIAS (black bars). (*−p<0.05 Student's t-test, error bars±2SE). Three independent experiments with different TT dose titrations (A, B, C) are shown.

FIG. 8 are graphical representations showing CMRF-44 and complement treated PBMC stimulate a reduced allogeneic naïve CD4⁺ T-lymphocyte reaction. Stimulators=irradiated overnight cultured PBMC treated with CMRF-44 and either AS (shaded bars) or HIAS (black bars). Responders=CD4⁺ CD45RA⁺ T-cells, 10⁵/well. (*−p<0.05 Student's t-test, error bars±2SE). Two independent experiments (A, B) are shown.

### DETAILED DESCRIPTION OF THE PREFERRED EMBODIMENTS

The present invention is predicated in part on the observation that the activity of an APC such as, for example, a dendritic cell, can be suppressed via the specific targeting of an activation antigen with an effective down-regulatory agent. Moreover, a specific down-regulatory agent may preferentially target a distinct sub-population of APCs. The targeted APC is thereby disabled or destroyed, leading to the potentially negative effects of such cells being reduced or prevented. The identification of the capability to specifically down-regulate targeted APCs enables applications as diverse as removing or reducing the rejection difficulties caused by host versus graft and graft versus host incompatibility, and ameliorating a range of auto-immune inflammatory reactions characterized by unwanted immune responses such as, for example, rheumatoid arthritis.

Accordingly, one aspect of the present invention contemplates a method for modulating the immuno-activity of an APC, said method comprising contacting said APC with an effective amount of an agent, which agent couples, binds or otherwise associates with a cell-surface activation molecule for a time and under conditions sufficient to prevent, inhibit or otherwise down-regulate one or more functional activities of said APC.

Reference herein to an “antigen-presenting cell” or “antigen-presenting cells” or its abbreviation “APC” or “APCs” refers to a cell or cells capable of endocytotic adsorption, processing and presenting of an antigen. The term “antigen presenting” means the display of antigen as peptide fragments bound to MHC molecules, on the cell surface. Many different kinds of cells may function as APCs including, for example, macrophages, B cells, follicular dendritic cells and dendritic cells.

An “antigen” is any organic or inorganic molecule capable of stimulating an immune response. The term “antigen” as used herein extends to any molecule such as, but not limited, to a peptide, polypeptide, protein, nucleic acid molecule, carbohydrate molecule, organic or inorganic molecule capable of stimulating an immune response.

One particularly useful APC in the context of the present invention is a dendritic cell. Dendritic cells are a population of widely distributed leucocytes that are highly specialized in antigen presentation via MHC II antigen or peptide complexes. They are the principal activators of resting T cells in vitro and a major source of immunogenic epitopes for specific T cell clones following the detection of an antigen in vivo or in vitro. As used herein, the term “dendritic cell” or “dendritic cells” (DC) refers to a dendritic cell or cells in its broadest context and includes any DC that is capable of antigen presentation. The term includes all DC that initiate an immune response and/or present an antigen to T-lymphocytes and/or provide T-cells with any other activation signal required for stimulation of an immune response.

Accordingly, another aspect of the present invention contemplates a method for modulating the immuno-activity of a DC, said method comprising contacting said DC with an effective amount of an agent, which agent couples, binds or otherwise associates with a cell surface activation molecule, for a time and under conditions sufficient to prevent, inhibit or otherwise down-regulate one or more functional activities of said DC.

Reference herein to “DC” should be read as including reference to cells exhibiting dendritic cell morphology, phenotype or functional activity and to mutants or variants thereof. The morphological features of dendritic cells may include, but are not limited to, long cytoplasmic processes or large cells with multiple fine dendrites. Phenotypic characteristics may include, but are not limited to, expression of one or more of MHC class I molecules, MHC class II molecules, CD1, CD4, CD11c and CD123. Functional activity includes, but is not limited to, a stimulatory capacity for naïve allogeneic T cells. “Variants” include, but are not limited to, cells exhibiting some but not all of the morphological or phenotypic features or functional activities of DC. “Mutants” include, but are not limited to, DC which are transgenic wherein said transgenic cells are engineered to express one or more genes such as genes encoding antigens, immune modulating agents or cytokines or receptors. Reference herein to a DC refers to both partially differentiated and fully differentiated DC and to activated and non-activated DC.

Without limiting the invention to any one theory or mode of action, two sub-populations of blood DC have been described, based on the differential expression of CD11c antigen and peanut agglutinin binding. They have distinctive characteristics and functions, including differential regulation by cytokines. The classical CD11c⁺ “myeloid” DC traffic into tissues and mucosal surfaces to act as immune sentinel cells and, after activation by pathogens or appropriate inflammatory stimuli, migrate via lymphatics to secondary lymphoid organs, where they initiate immune responses. The CD11c⁻ “lymphoid” DC express high levels of the CD123 antigen (interleukin-3 receptor a chain) on their surface. They are presumed to enter lymph nodes directly via the high endothelial venule to participate in immune responses. The CD11c⁺ blood DC express the CD13 and CD33 myeloid differentiation antigens and include precursors for both epithelial and deep tissue (e.g. dermal) DC. In contrast, the CD123^{hi} DC lack expression of CD13 and CD33 but express CD4 in greater amounts.

Still without wishing to limit the operation of the present invention to any one mode of action, it has been determined that the CD11c⁺ DC has the greater antigen uptake and immuno-stimulatory capacity, whereas the CD11c⁻ CD123^{hi} DC has the ability to produce substantial amounts of interferon-α upon stimulation with pathogens. In the context of the present invention, it is proposed that cells which have expressed a surface antigen, which expression occurs during and/or as a result of activation, may become preferred targets for agents capable of adversely affecting the continued viability of these cells. Hence, agents of the present invention may preferentially target a sub-population of DC, which express an activated antigen.

Preferably, the targeted DC is a myeloid DC and, even more preferably, belongs to the CD11c⁺ DC sub-population.

Accordingly, in a related embodiment of the present invention, there is provided a method for modulating the immuno-activity of a sub-population of DC, said method comprising contacting said sub-population with an effective amount of an agent, which agent couples, binds or otherwise associates with a cell-surface activation molecule, for a time and under conditions sufficient to prevent, inhibit or otherwise down-regulate one or more functional activities of said sub-population.

In an even more preferable embodiment of the present invention, there is provided a method for modulating the immuno-activity of a CD11⁺ c⁺ DC sub-population, said method comprising contacting said sub-population with an effective amount of an agent, which agent couples, binds or otherwise associates with a cell-surface activation molecule, for a time and under conditions sufficient to prevent, inhibit or otherwise down-regulate one or more functional activities of said CD11c⁺ DC sub-population.

A reference to an APC being “immuno-active”, or other forms thereof such as “immuno-activity”, is a reference to a range of in vivo or in vitro activities of APC, such as occurs in the context of an immune response. For example, immune activities contemplated herein include inter alia one or more of antigen endocytosis, antigen processing and/or presentation. In the context of the present invention, a preferred APC is a DC or, in particular, an activated CD11c⁺ sub-population thereof.

As detailed above, the range of immuno-activities potentially displayed by an APC encompasses and includes, inter alia, antigen endocytosis, processing and presentation, on contact with an agent capable of eliciting such a response. The modulation of such “immuno-activity”, therefore, refers to the ability to alter, suppress or increase, up- or down-regulate or otherwise affect the level and/or amount of APC immuno-activity. Preferably, the modulation results in suppression, inhibition or down-regulation of APC immuno-activity. In this context, modulating a cell's immuno-activity also encompasses and includes affecting the viability of the said cell or cells and, in a preferred embodiment, extends to their depletion, inactivation and/or eventual apoptosis.

The method of the present invention is performed by contacting an APC, and preferably a DC or sub-population thereof, with an “agent”, through which one or more functional activities of said APC is prevented, inhibited or otherwise down-regulated. As mentioned, the down-regulation may be as a result of inactivation of one or more APC activities and/or by depletion or lysis of said APC.

Reference herein to an “agent” should be understood as a reference to any proteinaceous or non-proteinaceous molecule which couples, binds or otherwise associates with the subject cell-surface activation molecule. The subject agent may be linked, bound or otherwise associated with any proteinaceous or non-proteinaceous molecule. For example, it may be associated with a molecule which permits targeting to a localized region. Said proteinaceous molecule may be derived from natural, recombinant or synthetic sources including fusion proteins or following, for example, natural product screening. Said non-proteinaceous molecule may be derived from natural sources such as, for example, natural product screening or may be chemically synthesized, or may be derived from high throughput screening of chemical libraries. Suitable agents that may have applicability in the instant invention include, for example, any protein comprising one or more immunoglobulin domains, and extend to antibodies within the immunoglobulin family of plasma proteins which includes immunoglobulin (Ig)A, IgM, IgG, IgD and IgE. The term “antibody” includes and encompasses fragments of an antibody such as, for example, a diabody, derived from an antibody by proteolytic digestion or by other means including but not limited to chemical cleavage. An antibody may be a “polyclonal antibody” or a “monoclonal antibody”. “Monoclonal antibodies” are antibodies produced by a single clone of antibody-producing cells. Polyclonal antibodies, by contrast, are derived from multiple clones of diverse specificity. The term “antibody” also encompasses hybrid antibodies, fusion antibodies and antigen-binding portions, as well as other antigen-binding proteins such as T-associated binding molecules.

The agent of the present invention may form a complex with a cell-surface activation molecule on an APC, by binding or otherwise associating with the said molecule via any suitable interactive bonding mechanism including, for example, non-covalent bonding such as ionic bonding or co-valent bonding. In a preferred embodiment, the cell-surface activation molecule is bound by an amount of antibody effective to form a complex under conditions which result in the prevention, inhibition or down-regulation of one or more functional activities of an APC and, in particular, a DC. An “effective amount” means an amount necessary to at least partly obtain the desired response, viz to prevent, inhibit or down-regulate one or more functional activities of an APC, or to increase or otherwise potentiate the onset of an appropriate inhibitory or down-regulatory response, or to induce or otherwise effect the depletion, lysis or malfunctioning of an APC.

By “cell-surface activation molecule” is meant a molecule the expression of which is up-regulated upon stimulation of an APC. For example, a DC may be activated upon exposure to a foreign antigen to which the generation of, an immune response is desirable. Furthermore, DC may be activated in other circumstances, such as where aberrant activation occurs in response to their exposure to a “self” molecule, thereby leading to the induction of an undesirable auto-immune response.

Accordingly, in a preferred embodiment of this aspect of the present invention, the agent comprises a monoclonal antibody (mAb) such as, for example, CMRF-44, or a derivative, fragment, homolog, analog or chemical equivalent or mimetic thereof and the cell-surface activation molecule extends to encompass derivatives, fragments, homologs, analogs or chemical equivalents or mimetics thereof, expressed on the surface of a DC.

Preferably, the DC is a CD11c⁺ DC.

“Derivatives” include fragments, parts, portions, mutants, variants and mimetics from natural, synthetic or recombinant sources including fusion proteins. Parts or fragments include, for example, active regions of an agent or cell-surface activation molecule. Derivatives may be derived from insertion, deletion or substitution of amino acids. Amino acid insertional derivatives include amino and/or carboxylic terminal fusions as well as intra-sequence insertions of single or multiple amino acids. Insertional amino acid sequence variants are those in which one or more amino acid residues are introduced into a predetermined site in the protein although random insertion is also possible with suitable screening of the resulting product. Deletion variants are characterized by the removal of one or more amino acids from the sequence. Substitutional amino acid variants are those in which at least one residue in the sequence has been removed and a different residue inserted in its place. An example of substitutional amino acid variants is conservative amino acid substitution. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences including fusions with other peptides, polypeptides or proteins.

Chemical and functional equivalents of the an agent or cell-surface activation molecule should be understood as molecules exhibiting any one or more of the functional activities of these molecules and may be derived from any source such as by being chemically synthesized or identified via screening processes such as natural product screening.

The derivatives of an agent or cell-surface activation molecule include fragments having particular epitopes or parts of the entire molecule fused to peptides, polypeptides or other proteinaceous or non-proteinaceous molecules.

Analogs of an agent or cell-surface activation molecule contemplated herein include, but are not limited to, modification to side chains, incorporating of unnatural amino acids and/or their derivatives during peptide, polypeptide or protein synthesis and the use of cross-linkers and other methods which impose conformational constraints on the proteinaceous molecules or their analogs.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acid contemplated herein is shown in Table 1.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmule |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl--aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl-Nmbc | | | |
| ethylamino)cyclopropane | | | |

Cross-linkers can be used, for example, to stabilize 3D conformations, using homobifunctional cross-linkers such as the bifunctional imido esters having (CH₂)ₙ spacer groups with n=1 to n=6, glutaraldehyde, N-hydroxysuccinimide esters and heterobifunctional reagents which usually contain an amino-reactive moiety such as N-hydroxysuccinimide and another group specific-reactive moiety.

To effectively prevent, inhibit or otherwise down-regulate an immuno-activity of an APC, by binding or associating with a cell-surface activation molecule, a range of approaches and conditions may be utilized. For example, an agent may be conjugated with another molecule. Such an agent-conjugate may comprise an antibody as hereinbefore described, linked via means such as chemical linkage, to another molecule such as but not limited to a peptide, polypeptide, protein, enzyme, nucleic acid molecule including an oligonucleotide, carbohydrate molecule or a polysaccharide molecule or radioactive atom. Antibody conjugates may in some circumstances, be more efficacious in causing the desired outcome. For example, an antibody may be conjugated with a toxic component so as to induce cellular inactivation and/or lysis upon (i.e. during or after) the formation of an antibody/cell-surface activation molecule complex on the surface of an APC. Methods for the conjugation of molecules such as, but not limited to, toxic molecules are well known it the art. In this embodiment of the invention, such antibody conjugates may directly induce inactivation and/or lysis of an APC.

To the extent that the agent is an antibody, an APC may undergo opsonization by the antibody thereby leading to the induction of one or more effector mechanisms, including uptake of opsonized DC by phagocytic cells (such as macrophages), which express an Fc receptor, or lysis of opsonized DC by killer cells such as, but not limited to, NK and K cells, which also express an Fc receptor. The latter process is known in the art as antibody-dependent cell-mediated cytotoxicity. Any conditions sufficient to result in the prevention, inhibition or down-regulate of one or more functional activities of an APC are suitable for the practice of the present invention, In yet another alternative, an agent of the present invention, in particular an antibody, may activate the complement system, triggering a complement-mediated lytic response.

Complement-mediated cytotoxicity or lysis is particularly suited to immuno-therapeutic applications where the depletion, down-regulation or destruction of specific cells is desirable. Where an agent such as an Ab is engaged by the complement system, chemical conjugation with toxic moieties becomes unnecessary. A very localized immune response, culminating in APC, such as DC, lysis, may result. Under most conditions, lysis is substantially restricted to the cell to which the agent binds and occurs without the necessity to conjugate a toxic moiety, the presence of which may increase the risk that cells other that target cells are concomitantly inadvertently affected.

In all instances, cytotoxicity requires that an agent recognize and bind, complex or otherwise associate with a cell-surface activation molecule. Preferably the agent comprises the mAb CMRF-44.

Without wishing to limit the invention to any one mode of action or practice, the particular nature of effector mechanism which is stimulated may determine the nature of the immuno-activity which is modulated as well as the type and extent of modulation effected. For example, an antibody conjugated with a highly toxic component may induce rapid lysis of an APC once bound to a targeted cell-surface activation molecule. Lysis may proceed directly and cellular debris may be removed by, for example, circulating macrophages. An antibody coupled to a less toxic molecule may have the effect of inhibiting the metabolic activity of an APC, causing it to be less able to process and present, or less efficient in processing and presenting, antigen. Alternatively, cell-mediated cytotoxicity may result in, for example, the ability of an APC to endocytose antigen being disrupted or prevented, or in the number of APC being depleted, or in the interruption of APC differentiation and/or activation.

Accordingly, depending on the particular conditions under which an agent such as a mAb associates with a cell-surface activation molecule, a functional activity of the said APC may be affected. Preferably the functional immuno-activity which is modulated is one or more of antigen endocytosis, antigen processing and/or presentation, elicited on contact of an antibody and or an antibody-conjugate with an antigen.

In a preferred method, modulation of immuno-activity of an APC is achieved via a mAb and, in particular, CMRF-44, and complement-mediated cytotoxicity. Preferably the APC is a DC.

Accordingly, the present invention in a preferred embodiment provides a method for modulating the immuno-activity of an APC, said method comprising contacting said APC with an effective amount of a mAb for a time and under conditions sufficient to prevent, inhibit or otherwise down-regulate one or more of antigen endocytosis, antigen processing and/or antigen presentation by said APC.

Preferably said monoclonal antibody is CMRF-44.

Still more preferably, the APC is a DC.

The method of the present invention is therapeutically beneficial in circumstances where inactivation of APC functional activity and, in particular, DC functional activity may be desirable. Such circumstances include those wherein an unwanted, aberrant or otherwise undesirable immune response is or has been elicited. An example is in procedures involving allogeneic grafts such as bone marrow transplantation and tissue and/or organ transplantation, where graft versus host and/or host versus graft incompatibility may result in host cell or transplant cell rejection, respectively. An “allogeneic graft” is a graft wherein the donor is of the same species as the recipient, but is MHC incompatible. Effector cells of an immuno-competent allograft may target host antigens processed and presented by donor DC or, alternatively, antigens derived from the allograft may be endocytosed, processed and presented by host DC to effector cells of the host's immune system, as hereinbefore described. In either case, the immune response comprises immuno-activity which directly or indirectly contributes to transplant and/or host tissue rejection.

The population of DC which are treated in accordance with the methods of the present invention may be located in vivo or in vitro and may comprise activated or differentiated DC. Generally, but not necessarily, activation of a sub-type of DC is concomitant with further cellular differentiation.

The agent of the present invention may, in one embodiment, be administered to a subject. Alternatively, sub-types within a population of DC isolated from a subject may be specifically destroyed or otherwise inactivated or rendered non-functional by contacting said sub-type in vitro with an effective amount of an agent, which agent couples, binds or otherwise associates with a cell-surface activation molecule, for a time and under conditions sufficient to prevent, inhibit or otherwise down-regulate one or more functional activities of said sub-type.

Preferably, the population of DC is within a subject.

Accordingly another aspect of the present invention is directed toga method for modulating an immune response in a subject, said method comprising administering to said subject an effective amount of an agent, which agent couples, binds or otherwise associates with an antigen presenting cell's surface activation molecule for a time and under conditions sufficient to prevent, inhibit or otherwise down-regulate one or more functional activities of said APC.

Preferably the APC is a CD11c⁺ DC.

Reference herein to cells of an “immuno-competent” allograft should be understood as a reference to a population of allograft cells which comprises immune cells. By “immune cells” is meant cells which directly or indirectly contribute to one or more aspects of an immune response, such as facilitating antigen presentation, phagocytosis, immune effector mechanisms, antibody dependent cytotoxicity, antibody production and cytokine production, inter alia, as hereinbefore defined.

Examples of immuno-competent allografts include bone marrow cells and spleen cells. Highly immature cells such as stem cells, which retain the capacity to differentiate into a range of immune or non-immune cell types, should also be understood to satisfy the definition of “immune cells” as utilized herein, due to their capacity to differentiate into immune cells under appropriate conditions. Accordingly, an allograft comprising stem cells is also an immuno-competent graft within the scope of the present invention. It should further be understood that, in the context of the present invention, an immuno-competent graft may also comprise a non-immune cell component. This would be expected where an unpurified bone narrow or spleen cell graft, for example, is the subject of transplantation, since such a graft may be expected to comprise red blood cells, fibroblasts, platelets, adipocytes and other such non-immune cells.

It should be understood that the allograft that is transplanted into a host may be in any suitable form. For example, the graft may comprise a population of cells existing as a single cell suspension or it may comprise a tissue sample fragment or an organ. The allograft may be provided by any suitable donor source. For example, the cells may be isolated from an individual or from an existing cell line. The tissue allograft may also be derived from an in vitro source such as a tissue sample or organ, which has been generated or synthesized its vitro.

A “subject” in the context of the present invention includes and encompasses mammals such as humans, primates and livestock animals (e.g. sheep, pigs, cattle, horses, donkeys); laboratory test animals such as mice, rabbits, rats and guinea pigs; and companion animals such as dogs and cats. Preferably, the mammal is a human.

A reduction in the presentation of an allograft antigen to host T cells or host antigen to donor T cells, as processed and presented by DC, has the potential to prevent or limit the extent of an immune response. This reduction in presentation may be achieved by, for example either down-regulation of antigen-processing or reducing or preventing antigen presentation. In this context, a “host” is synonymous with “subject” and includes a human subject, as well as other animals such as other mammals inter alia, as hereinbefore described.

Accordingly, another aspect of the present invention provides a method for down-regulating the immuno-activity of an immuno-competent graft, said method comprising administering to said subject an effective amount of an agent, which agent couples, binds or otherwise associates with an APC's surface activation molecule, for a time and under conditions sufficient to prevent, inhibit or otherwise down-regulate one or more functional activities of said APC.

Agents suitable for use in this aspect of the present invention include antibodies and, more particularly, monoclonal antibodies, as hereinbefore described. Preferably the mAb is CMRF-44. Preferably the subject is a human.

In a most preferred embodiment of the present invention, an agent comprising the mAb CMRF-44 or an appropriate functional derivative, homolog, analog, chemical equivalent or mimetic thereof, may be administered to a human subject undergoing allogeneic graft transplantation, such as bone marrow transplantation, in the expectation that the said mAb may locate, bind or otherwise associate with a cell-surface activation molecule of a donor antigen-presenting DC and hence down-regulate its function, thereby ameliorating or preventing the development of graft versus host disease.

Hence the methods of the present invention have application in the treatment and/or prophylaxis of conditions characterized by aberrant, unwanted or otherwise inappropriate immuno-activity of an allogeneic immuno-competent graft such as occurs in graft versus host disease. The incidence of graft versus host disease may be observed in any situation where an allogeneic immuno-competent graft is required to be transplanted into a host recipient, such as pursuant to treatment for certain forms of cancer wherein bone marrow transplants are necessitated.

Accordingly, in a preferred embodiment, the present invention provides a method for down-regulating the immuno-activity of a bone marrow graft in a subject, said method comprising administering to said subject an effective amount of mAb CMRF-44, for a time and under conditions sufficient to prevent, inhibit or otherwise down-regulate one or more more functional activities of said DC.

Reference to “down-regulating” the immuno-activity of an immuno-competent graft should be understood as a reference to at least partially down-regulating said activity. Without wishing to limit the present invention to any one theory or mode of action, it will be understood that down-regulation may be brought about under a range of different conditions. These include, for example, the utilization of an antibody-conjugate, the assistance of cells involved in cell-mediated cytotoxicity, and/or the involvement of the complement-mediated processes, as described hereinbefore, and the extent of down-regulation will be influenced by the nature of the conditions, inter alia.

In this context, an “effective amount” means an amount necessary to at least partly obtain the desired response, or to delay the onset or inhibit progression or halt altogether the onset or progression of a particular condition being treated. The amount varies depending upon the health and physical condition of the subject being treated, the taxonomic group of the subject being treated, the degree of protection desired, the formulation of the composition, the assessment of the medical situation and other relevant factors. It is expected that the amount will fall in a relatively broad range, which may be determined through routine trials.

Accordingly, another aspect of the present invention contemplates a method for the prophylactic and/or therapeutic treatment of a condition characterized by the aberrant, unwanted or otherwise inappropriate immuno-activity of an immuno-competent graft, said method comprising contacting said graft with an effective amount of an agent or a derivative, homolog, analog, chemical equivalent or mimetic thereof, which agent couples, binds or otherwise associates with an APC's surface activation molecule, for a time and under conditions sufficient to prevent, inhibit or otherwise down-regulate the immuno-activity of said APC.

Preferably the immuno-competent graft comprises allogeneic bone marrow cells.

Preferably the APC is a DC and the agent comprises the mAb CMRF-44.

More particularly, the present invention contemplates a method for the prophylactic and/or therapeutic treatment of a condition characterized by the aberrant, unwanted or otherwise inappropriate immuno-activity of an immuno-competent graft in a subject, said method comprising contacting said graft with an effective amount of an agent or a derivative, homolog, analog, chemical equivalent or mimetic thereof, which agent couples, binds or otherwise associates with an APC's surface activation molecule derived from said graft, for a time and under conditions sufficient to prevent, inhibit or otherwise down-regulate the said inappropriate immuno-activity of said graft.

Preferably, the said subject is a human. Preferably, the said condition is graft versus host disease.

Still more preferably said graft is an allogeneic bone marrow graft, spleen cell graft or a stem cell graft.

Reference herein to “therapeutic” and “prophylactic” treatment is to be considered in its broadest context. The term “therapeutic” does not necessarily imply that a subject is treated until total recovery Similarly, “prophylactic” does not necessarily mean that the subject will not eventually contract a disease condition. Accordingly, therapeutic and prophylactic treatment includes amelioration of the symptoms of a particular condition or preventing or otherwise reducing the risk of developing a particular condition. The term “prophylactic” may be considered as reducing the severity or the onset of a particular condition. “Therapeutic” may also reduce the severity of an existing condition.

The methods of the present invention may have further use in the prophylactic and/or therapeutic treatment of a range of other conditions characterized by an unwanted or undesirable immune response. Such conditions include, inter alia, those wherein the response is inappropriate as well as those wherein the response may be regarded as being physiologically normal but is nevertheless undesirable. Examples include auto-immune conditions, chronic inflammatory conditions, asthma and hypersensitivity, allergies to innocuous agents and transplant rejection.

More particularly, conditions which are proposed to be treatable using the methods of the present invention encompass auto-immune and inflammatory disorders such as, for example, rheumatoid arthritis, lupus erythematosus, systemic lupus erythematosus, Hashimotos thyroiditis, multiple sclerosis, myasthenia gravis, type 1 diabetes, anto-immune anaemia, thrombocytopenia, inflammatory bowel disease and Crohn's disease.

In any condition, where undesirable responses are triggered by the presentation of antigen, the methods of the present invention may find useful application.

Accordingly, another aspect of the present invention contemplates a method for the prophylactic and/or therapeutic treatment of a condition characterized by an aberrant, unwanted or otherwise inappropriate immune response in a subject, said method comprising administering to said subject an effective amount of an agent, which agent couples, binds or otherwise associates with an APC's surface activation molecule, for a time and under conditions sufficient to prevent, inhibit or otherwise down-regulate the immuno-activity of said APC.

The present invention further extends to pharmaceutical compositions and formulations comprising the said agents for use in conjunction with the instant methods. Such pharmaceutical compositions and formulations may be administered to a human or animal subject in any one of a number of conventional dosage forms and by any one of a number of convenient means. The agent of the pharmaceutical composition is contemplated to exhibit therapeutic activity when administered in an amount which depends on the particular case. The variation depends, for example, on the human or animal and the agent chosen. A broad range of doses may be applicable. Considering a patient, for example, from about 0.1 mg to about 1 mg of agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The agent may be administered in a convenient manner such as by the oral, intravenous (where water soluble), intraperitoneal, intramuscular, subcutaneous, intradermal or suppository routes or implanting (e.g. using slow release molecules). The agent may be administered in the form of pharmaceutically acceptable non-toxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered id tablet form, the tablet may contain a binder such as tragacanth, corn starch or gelatin; a disintegrating agent, such as alginic acid; and a lubricant, such as magnesium stearate.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant.

In accordance with these methods, the agent defined in accordance with the present invention may be co-administered with one or more other compounds or molecules. By “co-administered” is meant simultaneous administration in the same formulation or in two different formulations via the same or different routes or sequential administration by the same or different routes. For example, the subject agent may be administered together with an agonistic agent in order to enhance its effects. By “sequential” administration is meant a time difference of from seconds, minutes, hours or days between the administration of the two types of molecules. These molecules may be administered in any order.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of micro-organisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The prevention of the action of micro-organisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 30% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gun, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding a modulatory agent. The vector may, for example, be a viral vector.

The present invention further contemplates a combination of therapies, such as the administration to a subject of the agent of the present invention in a pharmaceutical composition or formulation together with a low dose of immuno-suppressive drugs.

Yet another aspect of the present invention is directed to the use of an agent of the present invention in the manufacture of a pharmaceutical composition or formulation for use in the method of the invention.

The present invention is further described by the following non-limiting Examples.

### EXAMPLE 1

### Material and Methods

CMRF-44 Purification

CMRF-44 (IgM) was purified from conditioned tissue culture supernatant (10% w/v FCS in RPMI 1640) by dilution in an equal volume of 0.15 mol/l Na₂HPOI₄, pH 7.2 and passage through a 2 ml column of Protein-L immobilized on agarose beads (Pierce #20510). The column was washed with the above buffer until eluent A₂₈₀ₙₘ<0.010. Bound material was eluted with 4 ml 0.1 mol/l glycine at pH 2.5 and immediately neutralized with 0.4 ml mol/l Tris at pH 9. The protein content was estimated by A₂₈₀ₙₘ measurement, it contained CMRF-44 immunoreactivity, and SDS-PAGE analysis under reducing conditions revealed only two bands with MW consistent with IgM H- and L-chains.

Cell Preparations: PBMC

PMBC were purified over Ficoll-Paque Plus (Pharmacia, Uppsala, Sweden) from buffy coats front volunteer donors by standard methods.

Purified Lineage Negative Blood DCs

Lineage negative cells were prepared from fresh PBMC MACS columns (Miltenyi Biotec, Becton Dickinson, Australia) and magnetic beads (Biomag, goat anti-mouse IgG Fc, Polysciences Inc., Warrington, Pa., USA) were prepared according to the manufacturer's protocols. Briefly, a 3-way stop-cock was attached to a large CS (6.3 ml) column, a 10 ml syringe filled with BSA/EDTA/PBS horizontally fitted to the stopcock, a 23 g needle inserted vertically and attached to the MACS (Vario) magnet. The end of the needle cover (attached to the needle) was clipped. The syringe was used to expel air from the needle and the column was washed by adding 35 ml of BSA/EDTA/PBS via the top of column. To prepare beads for addition to cells, beads were washed twice with cold 0.5% w/v BSA/2 mM EDTA/PBS (SACS buffer).

PBMC were stained with a prepared cocktail of monoclonal antibodies to enable removal of lineage positive cells. The lineage cocktail contained an optimized mix as follows: 25% v/v OKT3 (T cells, CD3); 15% v/v OKM1 (Mo, CD11b; 15% v/v CMRF31 (Mo, CD14); 10% v/v HUNK-2 (NK, CD16); 20% v/v FMC63 (B, CD19) All were IgG. Lin⁺ cell depleting mAb mix 0.1 ml was added per 10⁶ cells. The cells were mixed with the cocktail and incubated on ice with occasional shaking for 20 min. The preparation was washed twice with MACS buffer and were resuspended in washed magnetic beads (1 ml beads per 50×10⁶ cells). The cells were incubated on ice for 15 min with gentle mixing. The suspension was cleared initially on a MPC-1 magnet (Dynal, Carlton South, Victoria, Australia) and then the supernatant was passed through a BS depletion column (Miltenyi). The eluate was centrifuged for 5 min at 4° C., 500 g and resuspended in PBS. The eluted cells were lysed with Vitalize (BioErgonomics, St Paul, Minn.) to remove residual erythrocytes. To check for contaminating antibody-labelled cells, the preparations were stained with FITC-conjugated sleep anti-mouse immunoglobulin (FITC-SAM) (1:50, v/v) for 10 min. Lin⁻ cells were identified and collected on a FACS-Vantage cell sorter, FITC positive cells being gated out. To obtain DC sub-sets, CD11c-APC and CD 123-PE were added with the FITC-SAM and separated populations of CD11c⁺ and CD123^{hi} cells were sorted.

Complement Sources

Low-Tox-M Rabbit complement was obtained from Cedarlane Laboratories (Hornby, Ontario) Fresh serum (up to 24 hr), prepared by centrifugation of clotted blood, was used as autologous human complement.

PBMC Cytotoxicity Assay

PBMC (10 ml at 10⁷ cells/ml) were cultured in a 90 cm petri dish (Sarstedt, Ingle Farm, South Australia) at 37° C. overnight in 5% v/v. CO₂ to induce expression of CMRF-44 and CD83. After Ficoll separation to remove dead cells the cells were washed and resuspended in cytotoxicity medium (RPMI 1640, 0.3% w/v BSA, 25 mM Hepes). Aliquots of the cells were stained with either CMRF-44 or negative control IgM followed by FITC-SAM, CD14-PE and CD19-PE to check for upregulation of CMRF-44 antigen on DC. As some activated B-cells and monocytes, but not T- and NK cells, also express CMRF-44 antigen, DC were defined here as PE-negative, FITC-positive events. 1.2×10⁶ PBMC in 0.3 ml was added to each 5 ml polypropylene culture tube. Purified CMRF-44 (or control, TEPC-2 purified myeloma IgM, Sigma) was added at 20 ug/ml and the tubes were placed on ice for 20 min. Rabbit complement (50 μl) or 300 μl of either autologous human serum (AS) or heat activated autologous human serum (HIAS) was added and the tubes were cultured for 1 hr at 37° C. in a 5% v/v CO₂ incubator, followed by further washing. To monitor DC depletion, aliquots were stained with CD14/19-PE and with FITC conjugates of either the independent DC marker CD83 or control antibody, DC were defined as FITC⁺, PE⁻ flow cytometry events in the live forward scatter gate, and these were expressed as % of all cells in the live gate.

Purified DC (Lineage Negative) Cytotoxicity Assay

Purified DC (Lin⁻ cells) were cultured overnight with or without GM-CSF (200 U/ml, Schering-Plough, Sydney, NSW) and IL-3 (10 ng/ml, Invitrogen, Mulgrave, Victoria, Australia) in 0.5-1 ml of cytotoxicity medium, in round bottom polypropylene culture tubes (5 ml; Falcon, BD Biosciences, North Ryde, NSW). An aliquot taken before and after overnight culture was monitored for cell death by flow cytometry (Annexin-PE and PI). To assess the percentage of CMRF-44⁺ cells a portion of the cultured DC preparation was stained with biotinylated CMRF-44 or biotinylated IgM negative control followed by streptavidin-PE.Cy5 and either CD11c-FITC and HLA-DR-PE or CD123-PE and HLA-DR-FITC. To effect depletion, approximately 5×10⁴ cells in each tube were stained with or without CMRF-44 (20 μg/ml, as for PBMC, Initially cells were resuspended in 500 μl of cytotoxicity medium, 25 μl of rabbit complement was added and the cells were cultured at 37° C. as above for PBMC. Autologous human serum was used thereafter.

For DC subset analysis, Lin⁻ cells treated with CMRF-44 mAb and complement were stained with either CD11c-FITC and HLA-DR-PE (for Lin⁻ cells and CD11c purified cells) or with CD123-PE and HLA-DR-FITC (for CD123 purified cells). PI and Annexin-V were used to assess apoptosis in enriched DC preparations, otherwise 7-AAD was used to exclude dead cells.

Flow Cytometry DC Enumeration

TRUCOUNT tubes (BD Biosciences) were used to quantitate mAb and complement-mediated cell depiction. Purified Lin⁻ DCs (or CD11c⁺, or CD123^{hi} subsets) were cultured overnight with cytokines (GM-CSF and IL-3) in polypropylene tubes. Cells where washed twice in cytotoxicity medium. An aliquot of 100 μl, containing 20,000 cells was added to polypropylene tubes. 200 μl cytotoxicity medium was then added, then either 20 μl of medium or CMRF-44 or control IgM. Cells were incubated on ice for 30 min., centrifuged and 120 μl of supernatant removed. Serum or heat inactivated serum (200 μl) was added and tubes incubated at 37° C. for 1 hr. Cells were centrifuged and 300 μl supernatant removed. To the 100 μl remaining, antibodies were added and tubes incubated for 20 min on ice. PUS (220 μl) was then added. After this, 300 μl of cells was transferred to TruCOUNT tubes and vortexed. Cells were left for 10 min and revortexed before counting. Data were expressed as cells per 10,000 beads.

Functional Assays

For tetanus toxoid (TT) and keyhole limpet haemocyanin (KLH) antigen presentation assays, PBMC from freshly donated blood were cultured overnight and treated as described above for the PBMC cytotoxicity assay. The washed cells were resuspended in 5% AS serum in RPMI1640 containing manufacturer's recommended quantities of HEPES, pyruvate, non-essential amino acids, penicillin and streptomycin (Invitrogen), and introduced into wells, at 1-3×10⁵/well as required of a 96-well round bottom culture plate (Falcon) containing TT or KLH in the same medium (final volume=200 μl/well). Plates were cultured for 6 days at 37° C. in 5% v/v CO_{2,} then 1 μCi of ³H-thymidine (Amersham, Sydney, NSW) was added per well, and culture continued for a further 1 hours before harvesting (TomTec Mach III, Hamden, Conn.) and ³H-thymidine incorporation measurement by liquid scintillation spectroscopy (Wallac, Finland). T-cell proliferation is presented as counts per minute (CPM).

For the allogeneic mixed lymphocyte reaction (MLR), PBMC treated as above with CMRF-44 and AS or HIAS were irradiated (3000 cGy) and added to wells in a round bottom 96-well plate containing 10⁵ allogeneic CD4+ CD45RA+ T-cell responders. The latter were prepared from buffy coat derived PBMC by rosette purification with neuraminidase-treated sheep red cells (and AB serum), followed by negative selection by FACS after staining with PE-conjugated mAbs for CD8, CD14, CD16, CD19, CD34, CD45RO, CD56, and HLA-DR. The purified cells were >85% CD4+ CD45RA+. The plates were cultured for 4 days, ³H-thymidine labeled, and harvested 16 hours later, and analysed as above.

### EXAMPLE 2

### Expression of CMRF-44 on PBMC, Lineage Negative Cells and Purified DC Subsets

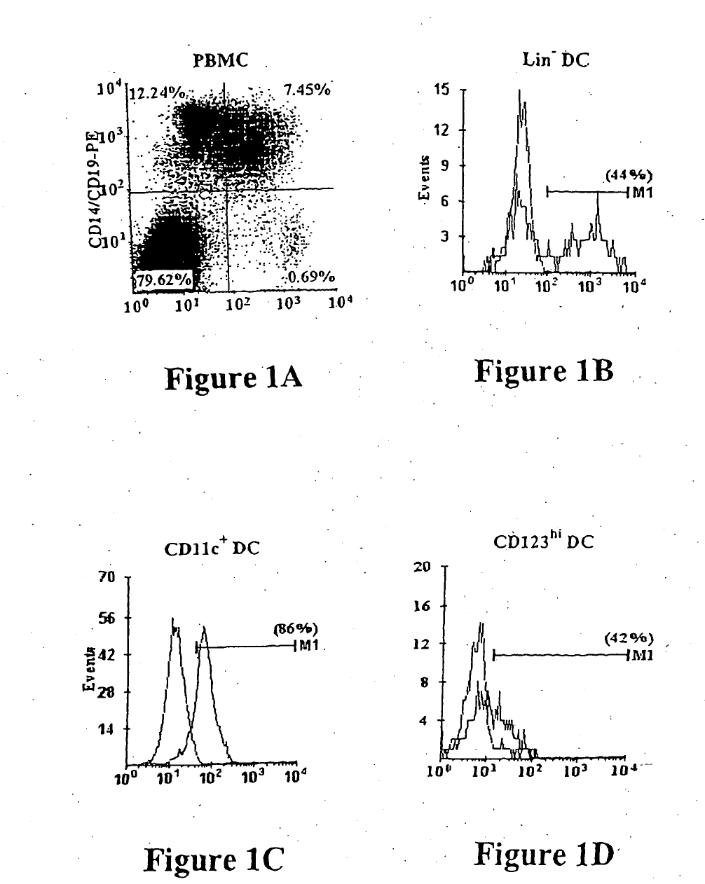

Repeated studies confirmed the presence of a small CMRF-44⁺ DC population in cultured PBMC (Fearnley et al., Blood 89: 3108-3716, 1997). As purified lineage negative blood DC populations are now routinely divided into CD11c and CD123 subsets, the expression of CMRF-44 on PBMC was analyzed, lineage negative and the CD11c and CD123 subsets. (FIG. 1) The CMRF-44 antigen is expressed on approximately 0.5-2.0% PBMC and on a high proportion of purified lineage negative DCs after culture. It was induced on the majority of CD11c⁺ DC and on a significant population of activated CD123^{hi} DC. These CMRF-44′ DC co-express the different DC activation antigen CD83 [Fearnley et al., 1999, supra].

### EXAMPLE 3

### CMRF-44 and complement Kills CD34⁺ Cells in PMBC

The cytotoxic effects of CMRF-44 and rabbit allogeneic and autologous complement were tested on PBMC DC populations, using a CD83 mAb to monitor the activated DC population.

Initial experiments with CMRF-44 and rabbit complement established that CMRF-44 mediated blood DC cytotoxicity. The effect titred with the antibody and occurred whether or not the cells were washed after incubation with antibody. Low concentrations (5% v/v) of rabbit complement were effective. However, despite being selected for its lack of spontaneous cytotoxicity of lymphoid cells, rabbit complement intermittently reduced the number of CD34⁺ cells, suggesting a background cytotoxic effect on blood DC. The CMRF-44 mAb and pooled AB serum as a complement source likewise mediated lysis of CD83⁺ cells but, again, donor variable background cytotoxicity was a problem.

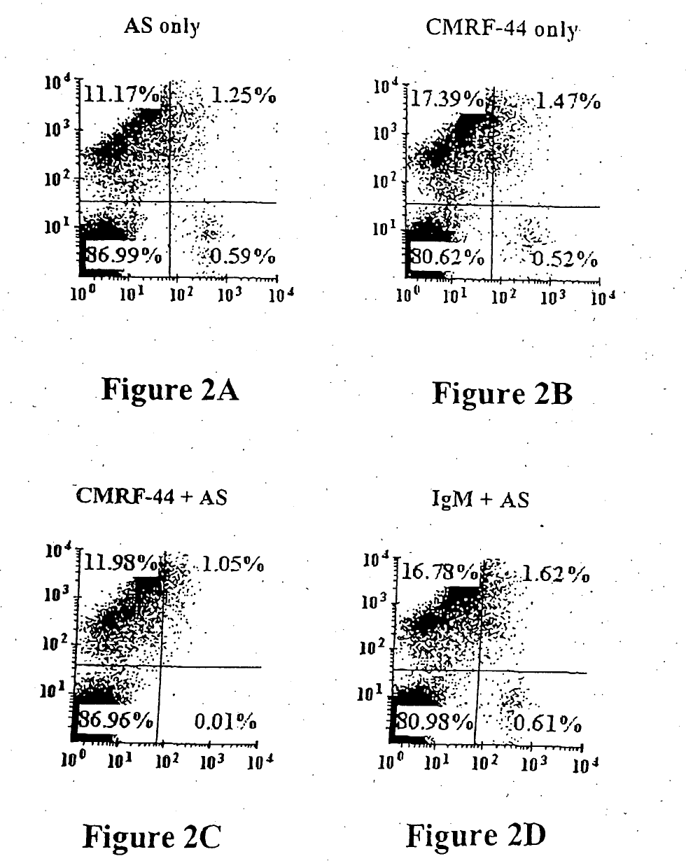

Autologous human serum (AS) was tested as a complement source (FIG. 2). This reduced background cytotoxicity to a consistently low level. No lysis occurred if the AS was heat inactivated (HIAS), nor did it occur if CMRF-44 was replaced by IgM negative control (FIG. 2D). Seven consecutive preparations were then analyzed: the mean percentage of CD14⁻/19⁻CD83⁺ cells in cultured PBMC treated with CMRF-44 and HIAS was 0.50% (SD=0.16.). CMRF-44 plus AS treatment reduced this to a mean of 0.06% (SD=0.08) (p<0.0005, Student's paired t-test). This and the data in FIG. 2 indicate that the cytotoxicity is highly specific.

### EXAMPLE 4

### Optimization of Cytotoxicity Assays Using Purified DC

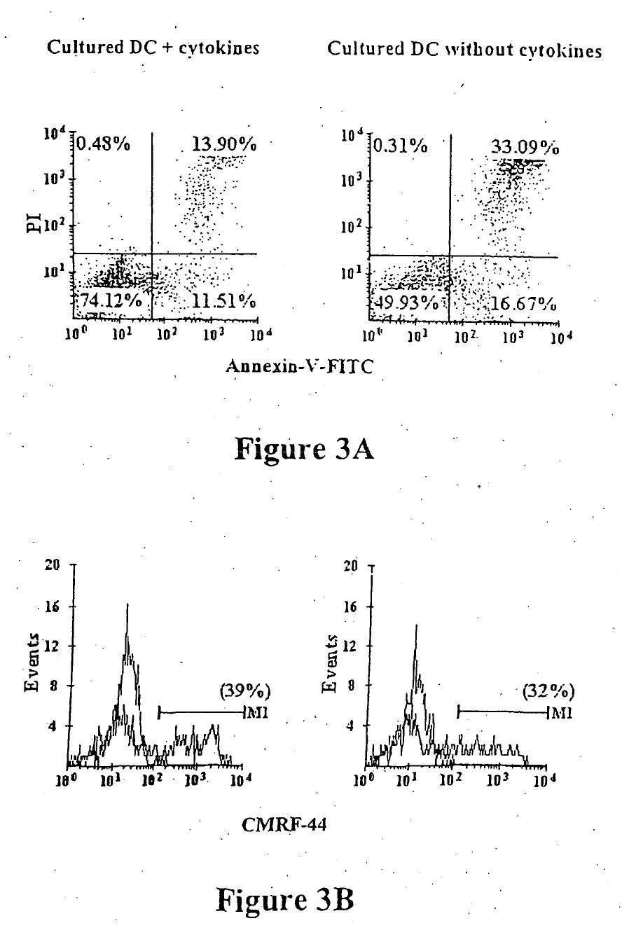

Blood DCs were purified from PBMC using negative immunoselection. Initial studies showed that a high proportion of DCs in these preparations underwent spontaneous cell death when cultured overnight, which contributed to a significant cytotoxicity background as measured by PI and Annexin-V staining. Other data indicated that the addition of cytokines would reduce background cytotoxicity of the CD123^{hi} DC subset in particular and, therefore, the Lin⁻ DCs were cultured in GM-CSF and IL-3 overnight. This reduced background apoptosis and cell death (FIG. 3A) and increased the proportion of CMRF-44⁺ cells available for analysis (FIG. 3B). Therefore, subsequent Lin⁻ DC preparations were routinely cultured overnight with GM-CSF and IL-3.

The optimal CMRF-44 mAb concentration for maximum cytotoxicity (measured both as a decrease in cells that were CD11c⁺ and HLA-DR⁺, and as an increase in total 7-AAD positive cells) was found to be greater than or equal to 10 μg/ml. The optimal AS concentration was found to be 1:2 v/v. These conditions were used in subsequent experiments.

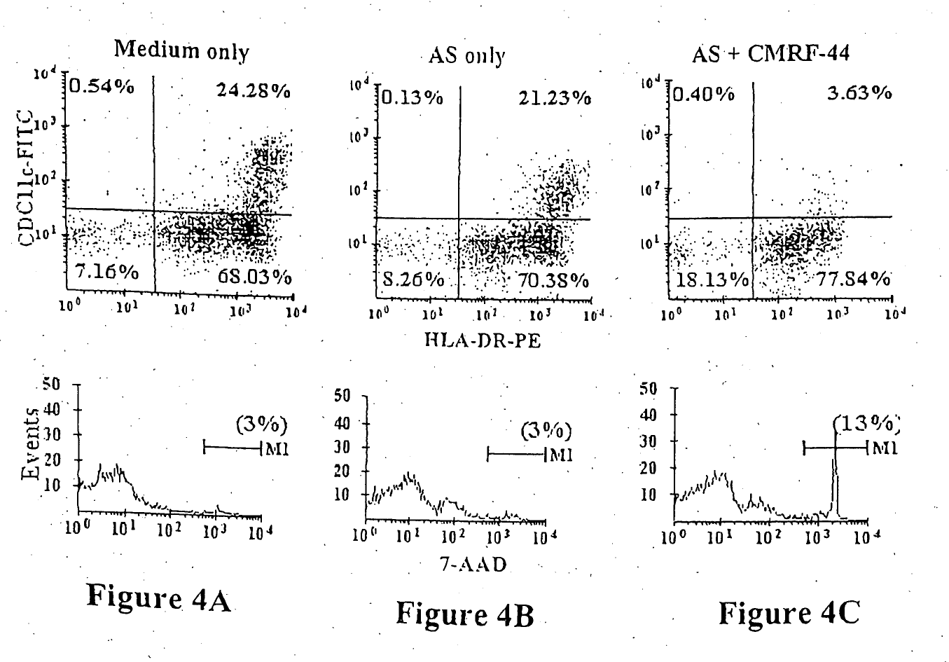

To investigate the subsets of Lin⁻ cells, which were susceptible to CMRF-44 mediated complement lysis, the Lin⁻ cells were stained with 7AAD, CD11c⁻ FITC and HLA-DR-PE. The results (FIG. 4) showed that the cells of CD11c⁺ population were profoundly reduced, accompanied by an increase in AAD positive cells. Optimization experiments, repeating the CMRF-44 titration and complement concentrations, confirmed these results.

The effect of CMRF-44 and complement on the CD123 subset within Lin⁻ cells was then examined. The results depended on the induction of the CMRF-44 antigen on this subset. Thus, in some cases the CD123⁺ (CD11c⁻) population was only partially affected (200%); in other cases a greater proportion (up to 90%) of CD 123⁺ cells was killed (Table 2).

TABLE 2
Percentage of CD11c⁺ DR⁺ and CD123⁺ in lineage negative
cells before and after treatment with CMRF-44.

| | Percentage of positive cells present | | | | | |
|---|---|---|---|---|---|---|
| Treatment of | | | % | | | % |
| Lin⁻ cells | CD11c | 7AAD | killed* | CD123 | 7AAD | killed* |
| CMRF-44 + | 1 | 32 | 92 | 3 | 36 | 90 |
| autologous | | | | | | |
| serum* | | | | | | |
| IgM + | 16 | 3 | | 30 | 6 | |
| autologous | | | | | | |
| serum* | | | | | | |
| CMRF-44 + | 16 | 6 | | 35 | 11 | |
| autologous | | | | | | |
| HI serum | | | | | | |
| Medium only | 22 | 7 | | 28 | 12 | |
| 1 in 1 | 17 | 3 | | 36 | 6 | |
| autologous | | | | | | |
| serum | | | | | | |
| 1 in 2 | 16 | 6 | | 35 | 11 | |
| autologous | | | | | | |
| HI serum | | | | | | |

*For % killed, compared IgM and CMRF-44 with autologous serum.

Cells were stained with either CD11c-FITC, HLA-DR-PE and 7 AAD or CD123-PE, HLA-DR-FITC and 7 AAD.

### EXAMPLE 5

### Absolute Counts to Document CMRF-44 Cytotoxic Effects

TruCOUNT bead methodology was introduced to monitor DC depletion accurately (see Example 1). This confirmed that both CD11c⁺ and CD11c⁻ (containing CD123^{hi}) populations were susceptible to CMRF-44 and AS treatment. An example is shown in Table 3.

TABLE 3
TruCOUNT analysis of CMRF-44 mediated cytotoxicity on lineage
negative sorted cells after overnight culture and treated with CMRF-44
and autologous serum.

| | No. of cells (events) per 10,000 beads in each quadrant | | | | |
|---|---|---|---|---|---|
| Treatment of | UL | UR | LL | LR | |
| Lin⁻ cells | (DR⁺11c⁻) | (DR⁺11c⁺) | (DR⁻11c⁻) | (DR⁻11c⁺) | Total |
| CMRF-44 + AS* | 280 | 111 | 272 | 56 | 819 |
| IgM + AS* | 347 | 573 | 373 | 63 | 1356 |
| CMRF-44 + HI | 370 | 452 | 254 | 74 | 1150 |
| AS | | | | | |
| Medium only | 472 | 474 | 195 | 11 | 1152 |
| AS only | 182 | 525 | 191 | 87 | 985 |
| HI AS only | 419 | 402 | 260 | 14 | 1095 |
| CMRF-44 only | 220 | 633 | 223 | 89 | 1165 |
| IgM (PEPC83) | 512 | 457 | 171 | 6 | 1146 |
| only | | | | | |

*Comparing CMRF-44 + AS with IgM + AS then 40% cells died. Most of the cells dying were CD11c⁺DR⁺.

Cultured lineage negative cells were treated with C F-44+AS, IgM+AS, CMRF-44+HI AS, medium only, autologous serum 1 in 2 (AS) only, heat inactivated (HI) AS only, CMRF-44 only, IgM only. 7AAD+ cells gated out. Cells stained with CD11cFITC, HLADR-PE and 7AAD. Cell count 46% cells stained CD11c⁺CMRF-44⁺.

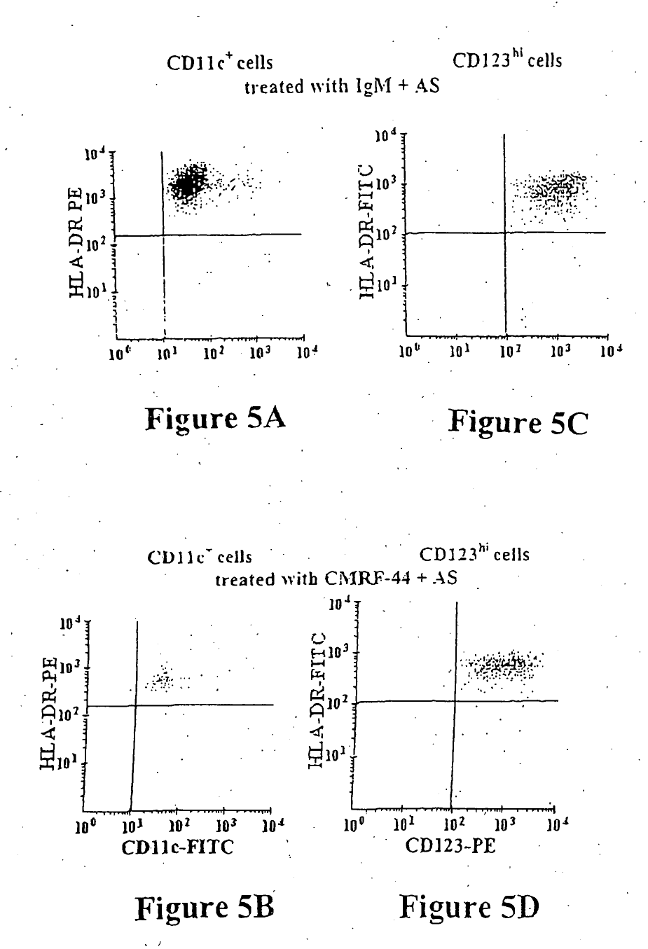

The two DC subsets were sort purified, cultured separately overnight with GM-CSF and IL-3 and then treated with CMRF-44 and AS. Purified CD11c⁺ DC were predominantly CMRF-44⁺ after culture and the majority (<90%) of these cells were depleted by treatment with CMRF-44 and AS. Purified CD123^{hi} DC were variably CMRF-44⁺ after culture, and, after treatment, this generally resulted in a lower percentage lysis compared to CD11c⁺ DC (n=3 experiments, Table 4, e.g, FIG. 5), but this percentage reflected near complete lysis of the CRMF-44⁺ CD123^{hi} DCs.

TABLE 4
Analysis of CMRF-44 and complement treated cultured CD11c⁺ and
CD123^{hi} DC

| | | No. of cells per 10,000 TruCOUNT beads | | | |
|---|---|---|---|---|---|
| | | Treatment | | | |
| Experiment | | | CMRF | % CMRF | % |
| No. | | IgM + AS | -44 + AS | -44⁺ | depletion |
| 1 | CD11c⁺ | 941 | 48 | 97% | 95% |
| | CD123^{hi} | 452 | 390 | 60% | 14% |
| 2 | CD11c⁺ | 6188 | 427 | 80% | 93% |
| | CD123^{hi} | 1870 | 1172 | 40% | 37% |
| 3 | CD11c⁺ | 4698 | 171 | 96% | 96% |
| | CD123^{hi} | 2129 | 471 | 72% | 78% |

Sort purified CD11c⁺ or CD123^{hi} DC were cultured overnight with GM-CSF+IL-3 and treated with 20 ug/ml of either negative control IgM or CMRF-44 followed by autologous serum 1:2 v/v (AS) as described in Example 1. Cells were then stained with 7-AAD and either CD11c-FITC and HLA-R-PE or CD 123-PE and HLA-DR-FITC. The 5^{th} column shows the % of CN-44+ cells (stained separately) prior to AS treatment. The 6^{th} column=100% [1−column 4/column 3].

### EXAMPLE 6

### Functional Effect of CMRF-44 and Complement DC Lysis on PBMC

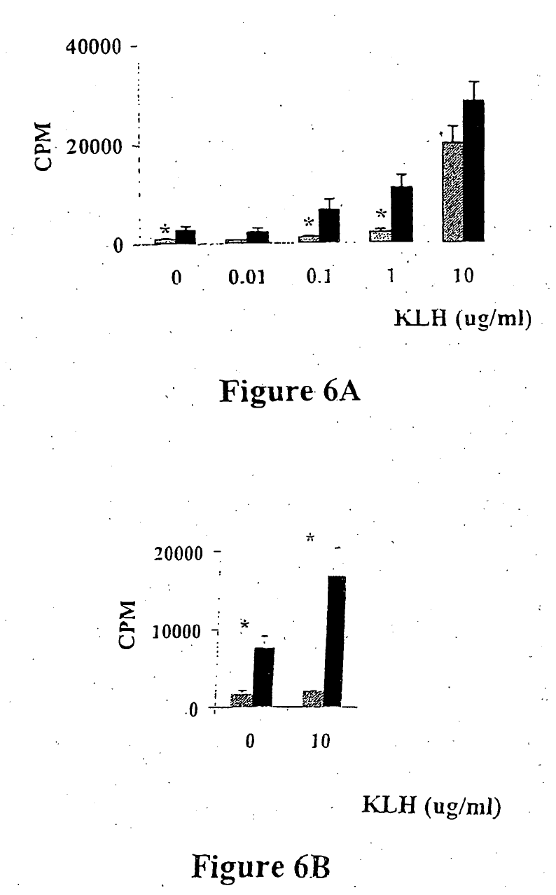

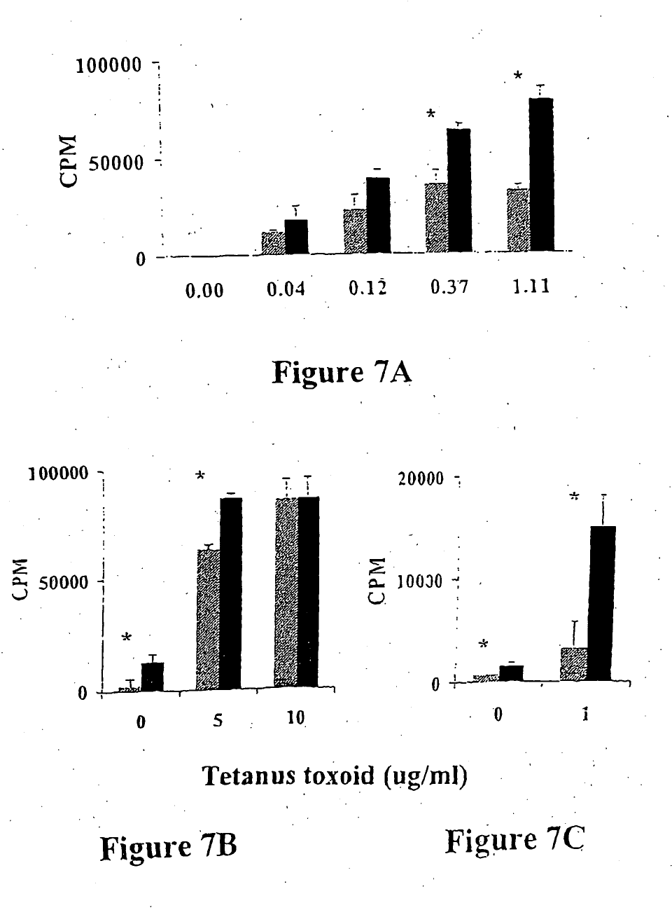

Previous experiments have shown that CMRF-44⁺ DC stimulate a recall tetanus toxoid (TT) proliferative T cell response and are essential to generate a primary (KLH) response. PBMC treated with CMRF-44 and AS were, therefore, tested for their ability to present TT and KLH. A substantial and statistically significant reduction in the ability of treated PBMC, relative to heat inactivated AS controls, to stimulate a primary proliferative response to KLH was found (p<0.05, FIG. 6). Reduced secondary responses to TT were also found, but were not as consistent or as marked (FIG. 7). Background counts were frequently significantly reduced after CMRF-44 and AS treatment, confirming the central role of CMRF-44⁺ cells in the autologous mixed lymphocyte reaction.

### EXAMPLE 7

### Stimulation of CD4⁺ T-Lymphocyte Reaction

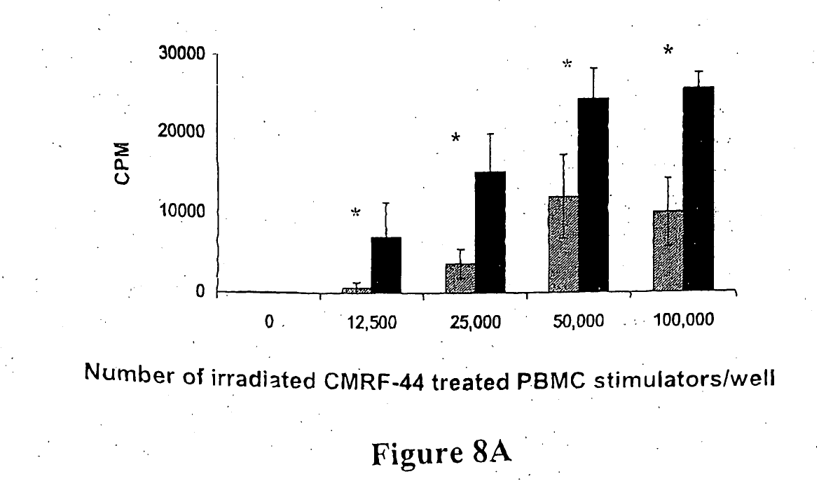

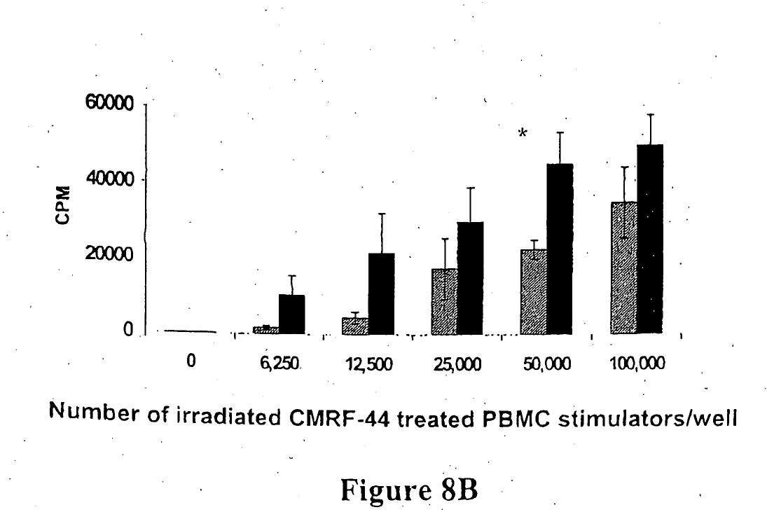

Irradiated overnight cultured PBMC depleted of DC using CMRF-44 and AS were then tested for their ability to stimulate allogeneic CD4⁺ CD45RA⁺ T-cells. Statistically significant reductions in T-cell proliferation were observed, compared to heat inactivated autologous serum controls. The inhibitory effect was most substantial at low stimulator: responder ratios. (FIG. 8).

The CMRF-44 mAb has, in continuation with autologous complement, specific cytotoxity activity against DC which undergo differentiation/activation in cultured blood PBMC, resulting in lysis of >88% of the CD11c DC subset associated with strong Tₕ1 responses. The CD123^{hi} DC subset, associated with Tₕ2 type responses but none-the-less capable of initiating a significant allogeneic response when activated, is also susceptible. These experiments establish the possibility of manipulating DC to prevent detrimental and to promote beneficial immune responses in allogeneic BMT and other forms of organ transplantation.

Those skilled in the art will appreciate that the present invention described herein is susceptible to variations and modifications other than those specifically described. It is to be understood that the present invention includes all such variations and modifications. The present invention also includes all of the steps, features, compositions and compounds referred to or indicated in this specification, individually or collectively, and any and all combinations of any two or more of said steps or features.

1. A method for modulating immuno-activity of an antigen presenting cell (APC) said method comprising contacting said cell with an effective amount of an agent which couples, binds or otherwise associates with a cell-surface activation molecule and in turn prevents, inhibits or otherwise down-regulates one or more functional activities of said cell.

2. The method of claim 1 wherein the APC is selected from a dendritic cell (DC), follicular DC, macrophage and B cell.

3. The method of claim 2 wherein the APC is a DC.

4. The method of claim 3 wherein the DC is a myeloid DC.

5. The method of claim 4 wherein the DC is a CD11c⁺ DC.

6. The method of claim 1 wherein the agent is an immunointeractive molecule.

7. The method of claim 6 wherein the immunointeractive molecule is an antibody or a functional equivalent thereof.

8. The method of claim 7 wherein the antibody is a monoclonal antibody or a functional equivalent thereof.

9. The method of claim 7 or 8 wherein the antibody or functional equivalent is specific for CMRF-44.

10. The method of claim 9 wherein the CMRF-44 antibody or its functional equivalent is capable of inducing lysis of the APC.

11. The method of claim 1 wherein the cell is mammalian derived.

12. The method of claim 11 wherein the mammalian cell is a human cell.

13. The method of claim 7 or 8 or 9 or 10 wherein the functional equivalent is a derivative, fragment, homolog, analog or chemical equivalent or mimetic of the antibody.

14. The method of claim 10 wherein lysis is caused by antibody-dependent cell-mediated cytotoxicity.

15. The method of claim 7 or 8 or 9 and 10 wherein the antibody in conjugated with a toxic component which kills or otherwise facilitates lysis of the APC.

16. A method for modulating the immuno-activity of an APC and/or lymphocyte, said method comprising contacting said APC with an effective amount of a monoclonal antibody to a cell surface antigen for a time and under conditions sufficient to induce lysis or cell death of said cell.

17. The method of claim 16 wherein the monoclonal antibody is specific for CMRF44 or its functional equivalent.

18. The method of claim 16 or 17 wherein the APC is DC.

19. The method of claim 18 wherein the DC is a CD11⁺ DC.

20. A method for modulating an immune response in a subject, said method comprising administering to said subject an effective amount of an agent, which agent couples, binds or otherwise associates with an APC surface activation molecule for a time and under conditions sufficient to prevent, inhibit or otherwise down-regulate one or more functional activities of said APC.

21. The method of claim 20 wherein the APC is DC.

22. The method of claim 21 wherein the DC is a CD11c⁺ DC.

23. The method of claim 20 wherein the agent is an antibody.

24. The method of claim 27 wherein the antibody is specific for CMRF44 or its functional equivalent.

25. A method for down-regulating the immuno-activity of an immuno-competent graft, said method comprising administering to said subject an effective amount of an antibody specific for CMRF-44 or its functional equivalent on an APC, for a time and under conditions sufficient to induce lysis or death of said APC.

26. A method for down-regulating the immuno-activity of a bone marrow graft in a subject, said method comprising administering to said subject an effective amount of an antibody specific for CMRF-44 or its functional equivalent on an APC for a time and under conditions sufficient to induce lysis or death of said APC.

27. A method for the prophylactic and/or therapeutic treatment of a condition characterized by the aberrant, unwanted or otherwise inappropriate immuno-activity of an immuno-competent graft, said method comprising contacting said graft with an effective amount of an antibody specific for CMRF-44 or its functional equivalent on an APC, for a time and under conditions sufficient to induce lysis or death of said APC.

28. The method of claim 25 or 26 or 27 wherein the APC is DC.

29. The method of claim 28 wherein the DC is a CD11c⁺ DC.

30. The method of claim 25 or 26 or 27 wherein the antibody is a monoclonal antibody.

31. Use of an antibody to CMRF-44 on an APC in the manufacture of a medicament for the treatment of an aberrant unwanted or otherwise inappropriate immuno-activity of an immune system.

32. Use of claim 31 wherein the APC is a DC.
